(19) United States
(12) Patent Application Publication
(10) Pub. No.: US 2024/0395367 A1
(43) Pub. Date: Nov. 28, 2024

(54) METHOD AND APPARATUS FOR DESIGNING LIGAND MOLECULES

(71) Applicant: Lemon Inc., Grand Cayman (KY)

(72) Inventors: Yuwei YANG, Los Angeles, CA (US); Siqi OUYANG, Beijing (CN); Hao ZHOU, Beijing (CN); Lei LI, Beijing (CN); Hang LI, Beijing (CN)

(21) Appl. No.: 18/694,101

(22) PCT Filed: Sep. 20, 2022
(86) PCT No.: PCT/SG2022/050675

§ 371 Date: Mar. 21, 2024

(30) Foreign Application Priority Data
Sep. 29, 2021 (CN) 202111154696.X

(51) Int. Cl. G16C 20/50; G06F 30/27

(52) U.S. Cl. CPC G16C 20/50; G06F 30/27

(57) ABSTRACT

According to the embodiments of the disclosure, a method, apparatus, device, storage media, and program products for designing ligand molecules is provided. The methods described herein include: editing a first molecular structure with an editing model to determine a second molecular structure, the editing at least comprising deleting a fragment from the first molecular structure or adding a fragment to the first molecular structure; in response to determining that an evaluation of the second molecular structure is better than the first molecular structure, training the editing model based on the editing, the evaluation at least indicating binding capacity between the second molecular structure and a target molecule; and determining a target structure of the ligand molecule for the target molecule with the trained editing model and based on the second molecular structure.

### CROSS REFERENCE

This application claims priority to the Chinese invention patent application entitled “method and apparatus for designing ligand molecules” filed on Sep. 29, 2021, with application No. 202111154696.X, the entirety of which is incorporated herein by reference.

### FIELD

The various implementations of the present disclosure relate to the field of computers, and more particularly, to a method, apparatus, device and computer storage media for designing ligand molecules.

### BACKGROUND

In drug discovery, an important task is to find small drug molecules (also known as ligand molecules, Ligand) that can effectively bind to target molecules (e.g., targeted protein molecules). In recent years, with the development of computer technology, computer-aided technologies such as Machine Learning have gradually been applied to the process of drug molecule discovery.

Traditional computer-aided techniques usually rely on experimental activity data. However, such experimental data are usually difficult to obtain, especially for new targeted proteins.

### SUMMARY

In a first aspect of the present disclosure, a method for designing ligand molecules is provided. The method includes: editing a first molecular structure with an editing model to determine a second molecular structure, the editing at least comprising deleting a fragment from the first molecular structure or adding a fragment to the first molecular structure; in response to determining that an evaluation of the second molecular structure is better than the first molecular structure, training the editing model based on the editing, the evaluation at least indicating binding capacity between the second molecular structure and a target molecule; and determining a target structure of the ligand molecule for the target molecule with the trained editing model and based on the second molecular structure.

In some embodiments, the editing model comprises an operation prediction model, and wherein editing a first molecular structure with an editing model comprises: determining, based on the first molecular structure, a set of first feature representations of a set of editable bonds in the first molecular structure; determining a target bond to be edited from the set of editable bonds and determining an editing operation to be applied to the target bond with the operation prediction model and based on the set of first feature representations; and editing the first molecular structure based on the determined editing operation.

In some embodiments, editing a first molecular structure comprising adding a fragment to the first molecular structure comprises: selecting a target fragment from a fragment library, wherein the fragment library comprises a plurality of three-dimensional fragments; and adding the target fragment to the first molecular structure.

In some embodiments, the plurality of three-dimensional fragments are constructed based on breaking single bonds in a set of drug molecules.

In some embodiments, the editing model comprises a fragment filtering model, and selecting a target fragment from a fragment library comprises: determining, based on the first molecular structure, a second feature representation of a target bond to be edited in the first molecular structure; and determining the target fragment from the fragment library with the fragment filtering model and based on the second feature representation.

In some embodiments, adding the target fragment to the first molecular structure comprises: determining an added bond added to the first molecular structure in the target fragment; and adding the target fragment to the first molecular structure based on the added bond.

In some embodiments, the editing model comprises a bond selection model, and wherein determining an added bond added to the first molecular structure in the target fragment comprises: determining a set of third feature representations of a set of candidate bonds that are capable of being used to add to the first molecular structure in the target fragment; determining a fourth feature representation based on the first molecular structure and the third feature representation; and determining the added bond from the set of candidate bonds with the bond selection model and based on the fourth feature representation.

In some embodiments, adding the target fragment to the first molecular structure based on the added bond comprises: determining a first pair of atoms associated with a target bond to be edited in the first molecular structure; determining a second pair of atoms associated with the added bond in the target fragment; determining a dihedral angle for adding the target fragment based on the first pair of atoms and the second pair of atoms; and adding the target fragment to the first molecular structure based on the determined dihedral angle.

In some embodiments, the editing model comprises an angle prediction model, and determining a dihedral angle for adding the target fragment comprises: determining a fifth feature representation associated with the first pair of atoms and the second pair of atoms based on the first molecular structure and the target fragment; and determining the dihedral angle for adding the target fragment with the angle prediction model and based on the fifth feature representation.

In some embodiments, editing a first molecular structure comprising deleting a fragment from the first molecular structure comprises: determining a bond to be deleted in the first molecular structure; and deleting a fragment associated with the bond to be deleted from the first molecular structure.

In some embodiments, determining a target structure of the ligand molecule for the target molecule comprises: processing the second molecular structure with the trained editing model to determine a third molecular structure; and determining the target structure of the ligand molecule based on the third molecular structure.

In some embodiments, the first molecular structure is generated by applying the first number of editing operations to an initial molecular structure, and determining a target structure of the ligand molecule for the target molecule comprises: incrementing the first number to determine the second number; and if the second number reaches a predetermined threshold, determining the second molecular structure as the target structure.

In some embodiments, the method further comprising: in response to determining that the evaluation of the second molecular structure is worse than or equal to the first molecular structure, determining, based on the evaluation, a probability that the second molecular structure is used to determine the target structure of the ligand molecule.

In some embodiments, the first molecular structure is generated by applying the first number of editing operations to an initial molecular structure, and the probability is further based on the first number.

In some embodiments, the evaluation is a first evaluation and training the editing model based on the editing comprises: training the editing model based on an optimization objective to be determined based on a difference between the first evaluation and a second evaluation of the first molecular structure.

In some embodiments, the editing model comprises a first graph model, and wherein editing a first molecular structure with an editing model comprises: generating a first graph based on the first molecular structure, wherein a first set of nodes in the first graph corresponds to a set of atoms in the first molecular structure, and a first set of edges in the first graph corresponds to a set of bonds in the first molecular structure; processing the first graph with the first graph model to determine a set of atomic-level features corresponding to the set of atoms; and editing the first molecular structure based on the set of atomic-level features.

In some embodiments, the editing model further comprises a second graph model, and editing the first molecular structure based on the set of atomic-level features comprises: determining, based on the set of atomic-level features, a first set of node features corresponding to a set of fragments in the first molecular structure and a first set of edge features corresponding to a set of bonds among the set of fragments; constructing a second graph based on the first set of node features and the first set of edge features; processing the second graph with the second graph model to determine a set of fragment-level node features corresponding to the set of fragments; and editing the first molecular structure based on at least one of the set of fragment-level node features or the set of atomic-level features.

In some embodiments, editing the first molecular structure based on at least one of the set of fragment-level node features or the set of atomic-level features comprises: determining, based on the set of fragment-level node features, a set of fragment-level edge features corresponding to a set of bonds among the set of fragments; and editing the first molecular structure based on at least one of the set of fragment-level node features, the set of atomic-level features, or the set of fragment-level edge features.

In some embodiments, the evaluation is further based on: drug-like QED of the second molecular structure; or synthesizability of the second molecular structure.

In a second aspect of the present disclosure, an apparatus for designing ligand molecules is provided. The apparatus includes: an editing module configured to edit a first molecular structure with an editing model to determine a second molecular structure, the editing at least comprising deleting a fragment from the first molecular structure or adding a fragment to the first molecular structure; and a training module configured to, in response to determining that an evaluation of the second molecular structure is better than the first molecular structure, train the editing model based on the editing, the evaluation at least indicating binding between the second molecular structure and a target molecule, wherein the editing module is further configured to determine a target structure of the ligand molecule for the target molecule with the trained editing model and based on the second molecular structure.

In some embodiments, the editing model includes an operation prediction model, and the editing module is further configured for: determining, based on the first molecular structure, a set of first feature representations of a set of editable bonds in the first molecular structure; determining a target bond to be edited from the set of editable bonds and determining an editing operation to be applied to the target bond with the operation prediction model and based on the set of first feature representations; and editing the first molecular structure based on the determined editing operation.

In some embodiments, the editing module is further configured for: selecting a target fragment from a fragment library, wherein the fragment library comprises a plurality of three-dimensional fragments; and adding the target fragment to the first molecular structure.

In some embodiments, the plurality of three-dimensional fragments are constructed based on breaking single bonds in a set of drug molecules.

In some embodiments, the editing model includes a fragment filtering model, and the editing module is further configured for: determining, based on the first molecular structure, a second feature representation of a target bond to be edited in the first molecular structure; and determining the target fragment from the fragment library with the fragment filtering model and based on the second feature representation.

In some embodiments, the editing module is further configured for: determining an added bond added to the first molecular structure in the target fragment; and adding the target fragment to the first molecular structure based on the added bond.

In some embodiments, the editing model includes a bond selection model, and the editing module is further configured for: determining a set of third feature representations of a set of candidate bonds that are capable of being used to add to the first molecular structure in the target fragment; determining a fourth feature representation based on the first molecular structure and the third feature representation; and determining the added bond from the set of candidate bonds with the bond selection model and based on the fourth feature representation.

In some embodiments, the editing module is further configured for: determining a first pair of atoms associated with a target bond to be edited in the first molecular structure; determining a second pair of atoms associated with the added bond in the target fragment; determining a dihedral angle for adding the target fragment based on the first pair of atoms and the second pair of atoms; and adding the target fragment to the first molecular structure based on the determined dihedral angle.

In some embodiments, the editing model includes an angle prediction model, and the editing module is further configured for: determining a fifth feature representation associated with the first pair of atoms and the second pair of atoms based on the first molecular structure and the target fragment; and determining the dihedral angle for adding the target fragment with the angle prediction model and based on the fifth feature representation.

In some embodiments, the editing module is further configured for deleting a fragment from the first molecular structure comprising: determining a bond to be deleted in the first molecular structure; and deleting a fragment associated with the bond to be deleted from the first molecular structure.

In some embodiments, the editing module is further configured for: processing the second molecular structure with the trained editing model to determine a third molecular structure; and determining the target structure of the ligand molecule based on the third molecular structure.

In some embodiments, the first molecular structure is generated by applying the first number of editing operations to an initial molecular structure, and the editing module is further configured for: incrementing the first number to determine the second number; and if the second number reaches a predetermined threshold, determining the second molecular structure as the target structure.

In some embodiments, the training module is further configured for: in response to determining that the evaluation of the second molecular structure is worse than or equal to the first molecular structure, determining, based on the evaluation, a probability that the second molecular structure is used to determine the target structure of the ligand molecule.

In some embodiments, the first molecular structure is generated by applying the first number of editing operations to an initial molecular structure, and the probability is further based on the first number.

In some embodiments, the evaluation is a first evaluation, and the training module is further configured for: training the editing model based on an optimization objective to be determined based on a difference between the first evaluation and a second evaluation of the first molecular structure.

In some embodiments, the editing model includes a first graph model, and the editing module is further configured for: generating a first graph based on the first molecular structure, wherein a first set of nodes in the first graph corresponds to a set of atoms in the first molecular structure, and a first set of edges in the first graph corresponds to a set of bonds in the first molecular structure; processing the first graph with the first graph model to determine a set of atomic-level features corresponding to the set of atoms; and editing the first molecular structure based on the set of atomic-level features.

In some embodiments, the editing model further includes a second graph model, and the editing module is further configured for: determining, based on the set of atomic-level features, a first set of node features corresponding to a set of fragments in the first molecular structure and a first set of edge features corresponding to a set of bonds among the set of fragments; constructing a second graph based on the first set of node features and the first set of edge features; processing the second graph with the second graph model to determine a set of fragment-level node features corresponding to the set of fragments; and editing the first molecular structure based on at least one of the set of fragment-level node features or the set of atomic-level features.

In some embodiments, the editing module is further configured for: determining, based on the set of fragment-level node features, a set of fragment-level edge features corresponding to a set of bonds among the set of fragments; and editing the first molecular structure based on at least one of the set of fragment-level node features, the set of atomic-level features, or the set of fragment-level edge features.

In some embodiments, the evaluation is further based on: drug-like QED of the second molecular structure; or synthesizability of the second molecular structure.

In a third aspect of the present disclosure, an electronic device is provided, comprising: a memory and a processor; wherein the memory is configured to store one or more computer instructions, wherein the one or more computer instructions, when executed by the processor, implement the method according to the first aspect of the present disclosure.

In a fourth aspect of the present disclosure, there is provided a computer-readable storage medium having one or more computer instructions stored thereon, wherein the one or more computer instructions, when executed by a processor, implement the method according to the first aspect of the present disclosure.

In a fifth aspect of the present disclosure, there is provided a computer program product comprising one or more computer instructions, wherein the one or more computer instructions, when executed by a processor, implement a method according to a first aspect of the present disclosure.

According to various embodiments of the present disclosure, it is possible to efficiently construct ligand molecules based on a self-supervised method, thereby improving the universality of the method.

### BRIEF DESCRIPTION OF THE DRAWINGS

The above and other features, advantages and aspects of the various embodiments of the present disclosure will become more apparent in conjunction with the accompanying drawings and with reference to the following detailed description. In the drawings, like or similar reference numerals denote like or similar elements, wherein:

FIG. 1 shows a schematic block diagram of a computing device capable of implementing some embodiments of the present disclosure;

FIG. 2 shows a schematic block diagram of a design module according to some embodiments of the present disclosure;

FIG. 3 shows a schematic block diagram of a fragment constructing module according to some embodiments of the present disclosure;

FIG. 4 shows a schematic block diagram of an editing module according to some embodiments of the present disclosure;

FIG. 5 shows a schematic block diagram of a feature extraction module according to some embodiments of the present disclosure; and

FIG. 6 shows a flowchart of an example method for designing ligand molecules according to some embodiments of the present disclosure.

### DETAILED DESCRIPTION

The following will describe embodiments of the present disclosure in more detail with reference to the accompanying drawings. Although certain embodiments of the present disclosure are displayed in the drawings, it should be understood that the present disclosure can be implemented in various forms and should not be construed as limited to the embodiments set forth herein. On the contrary, these embodiments are provided for a more thorough and complete understanding of the present disclosure. It should be understood that the accompanying drawings and embodiments of the present disclosure are for illustrative purposes only and are not intended to limit the scope of protection of the present disclosure.

In the description of embodiments of the present disclosure, the term “comprising” and similar terms should be understood as open-ended inclusion, that is, “including but not limited to”. The term “based on” should be understood as “at least partially based on”. The term “one embodiment” or “the embodiment” should be understood as “at least one embodiment”. The terms “first”, “second”, etc. may refer to different or identical objects. The following text may also include other explicit and implicit definitions.

As discussed above, with the development of computer technology, computer-aided technologies such as Machine Learning are gradually being applied to the process of drug molecule discovery. Traditional computer-aided technologies usually rely on experimental activity data. However, such experimental data is usually difficult to obtain, especially for new targeted proteins. This results in the limited scope of application of traditional computer-aided drug discovery.

According to the implementations of the present disclosure, a scheme for designing ligand molecules is provided. In this scheme, a first molecular structure may be edited with an editing model to determine a second molecular structure, where the editing at least includes deleting a fragment from the first molecular structure or adding a fragment to the first molecular structure. Furthermore, in response to determining that an evaluation of the second molecular structure is better than the first molecular structure, the editing model is trained based on the editing, where the evaluation at least indicates binding capacity between the second molecular structure and the target molecule. Furthermore, a target structure of the ligand molecule for the target molecule may be determined with the trained editing model and based on the second molecular structure.

By using the editing model to predict a relationship between fragments, and to obtain a better evaluation based on the editing to train the editing model, the embodiments of the present disclosure may be based on a self-supervised method to effectively construct ligand molecules, thereby improving the universality of the method.

The following describes the basic principles and several example implementations of the present disclosure with reference to the accompanying drawings.

### Example Device

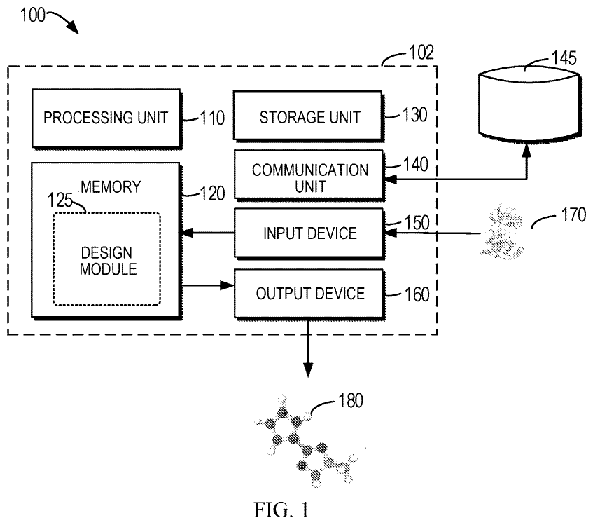

FIG. 1 shows a schematic block diagram of an example device 100 that can be used to implement embodiments of the present disclosure. It should be understood that the device 100 shown in FIG. 1 is merely exemplary and should not constitute any limitation on the functionality and scope of the implementations described in this disclosure. As shown in FIG. 1, the components of the device 100 may include but are not limited to one or more processors or processing units 110, a memory 120, a storage device 130, one or more communication units 140, one or more input devices 150, and one or more output devices 160.

In some implementations, the device 100 can be implemented as various user end points or service end points. Service end points can be servers, large computing devices, etc. provided by various service providers. User end points are any type of mobile end point, fixed end point, or portable end point, including mobile phones, multimedia computers, multimedia tablets, internet nodes, communicators, desktop computers, laptop computers, notebook computers, netbook computers, tablet computers, personal communication system (PCS) devices, personal navigation devices, personal digital assistants (PDAs), audio/Mobile Pentium 4, digital cameras/camcorders, positioning devices, television receivers, radio broadcast receivers, e-book devices, gaming devices, or any combination thereof, including accessories and peripherals of these devices or any combination thereof. It is also foreseeable that the device 100 can support any type of user-specific interface (such as “wearable” circuits, etc.).

The processing unit 110 may be an actual or virtual processor and is capable of performing various processing according to programs stored in the memory 120. In a multiprocessor system, multiple processing units execute computer-executable instructions in parallel to improve the parallel processing capability of the device 100. The processing unit 110 may also be referred to as a central processing unit (CPU), a microprocessor, a controller, or a microcontroller.

The device 100 typically includes multiple computer storage media. Such media can be any available media accessible to the device 100, including but not limited to volatile and nonvolatile media, removable and non-removable media. The memory 120 can be volatile memory (such as registers, caches, random access memory (RAM)), nonvolatile memory (such as read-only memory (ROM), electrically erasable programmable read-only memory (EEPROM), flash memory), or some combination thereof. The memory 120 can include one or more design modules 125 that are configured to perform various implemented functions described herein. The design module 125 can be accessed and operated by the processing unit 110 to implement corresponding functions. The storage device 130 can be removable or non-removable media and can include machine-readable media that can be used to store information and/or data and can be accessed within the device 100.

The functions of the components of the device 100 can be implemented in a single computing cluster or multiple computing machines, which can communicate through communication connections. Therefore, the device 100 can operate in a networked environment using logical connections with one or more other servers, personal computers (PCs), or another general network node. The device 100 can also communicate with one or more external devices (not shown) through the communication unit 140 as needed. External devices such as the database 145, other storage devices, servers, display devices, etc. can communicate with one or more devices that enable users to interact with the device 100, or with any device (such as network interface cards, modems, etc.) that enables the device 100 to communicate with one or more other computing devices. Such communication can be performed via input/output (I/O) interfaces (not shown).

The input device 150 may be one or more of various input devices, such as a mouse, keyboard, trackball, voice input device, camera, etc. Output device 160 may be one or more output devices, such as a display, speaker, printer, etc.

In some implementations, as shown in FIG. 1, the device 100 may, for example, receive an identification corresponding to a target molecule 170 (e.g., a targeted protein molecule) through the input device 150. For example, a user may input a PDB file through the input device 150 to indicate the corresponding target molecule 172.

In some implementations, the design module 125 may iteratively edit the molecular structure with the editing model to determine the target structure of the final ligand molecule 180. The process for determining the target structure of ligand molecule 180 will be described in detail below.

### Ligand Molecule Design

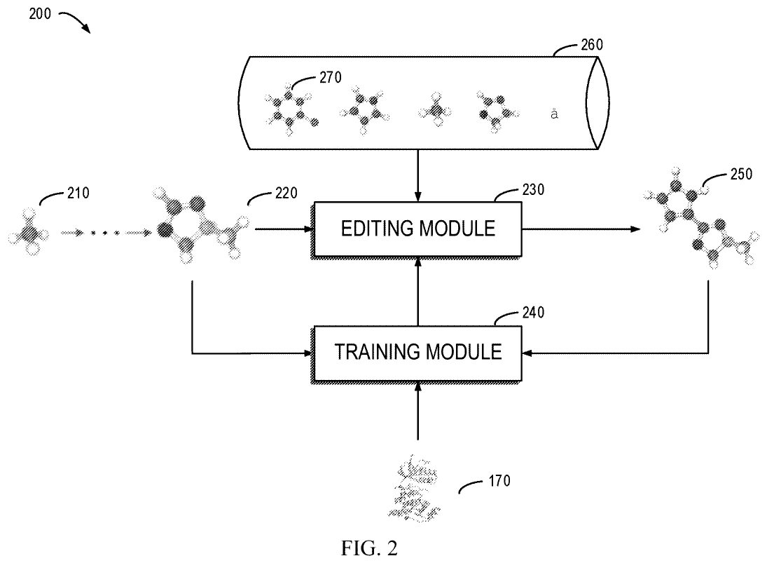

Referring first to FIG. 2, which illustrates a block diagram of a design module 125 according to some embodiments of the present disclosure. As shown in FIG. 2, the design module 125 includes a plurality of modules for implementing the process of designing ligand molecules according to some embodiments of the present disclosure. As shown in FIG. 2, the design module 125 includes an editing module 230 and a training module 240.

In some embodiments, the editing module 230 may edit a first molecular structure 220. Specifically, the editing may include deleting a fragment from the first molecular structure 220, such editing is also referred to as “delete editing operation”. Alternatively, editing may also include adding a new fragment to the first molecular structure 220, such editing is also referred to as “add editing operation”.

For the “delete editing operation”, the editing module 230 may determine a bond to be deleted in the first molecular structure 220 and accordingly delete a fragment associated with the bond to be deleted from the first molecular structure. For example, the editing module 230 may delete a group associated with the bond to be deleted from the first molecular structure and correspondingly add a hydrogen atom to construct a new molecular structure.

For the “add editing operation”, the editing module 230 may determine the bond to be edited in the first molecular structure 220 and accordingly select a fragment from a fragment library 260 to attach to the first molecular structure 220. During the “add editing operation”, the hydrogen atoms associated with the bond to be edited in the first molecular structure 220 and the hydrogen atoms corresponding to the selected fragment may be deleted accordingly to construct a new molecular structure.

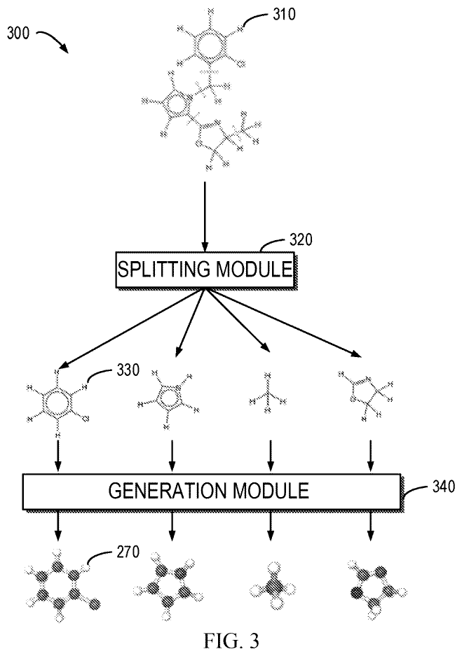

In some embodiments, the fragment library 260 may include a plurality of fragments 270. As shown in FIG. 2, such fragments 270 may be three-dimensional (3D) structural fragments. In some embodiments, the plurality of fragments 270 may be determined, for example, based on experimental knowledge. Alternatively, the plurality of fragments 270 may also be constructed based on existing drug molecules. The construction process of the fragment library 260 will be described in detail below with reference to FIG. 3.

In some embodiments, the first molecular structure 220 may be obtained, for example, from an initial molecular structure (e.g., methane molecule CH₄ shown in FIG. 2) through at least one editing process as discussed above. Alternatively, the first molecular structure 220 may also be an initial molecular structure, for example, which may be randomly selected by the editing module 230 or determined by the editing module 230 based on input.

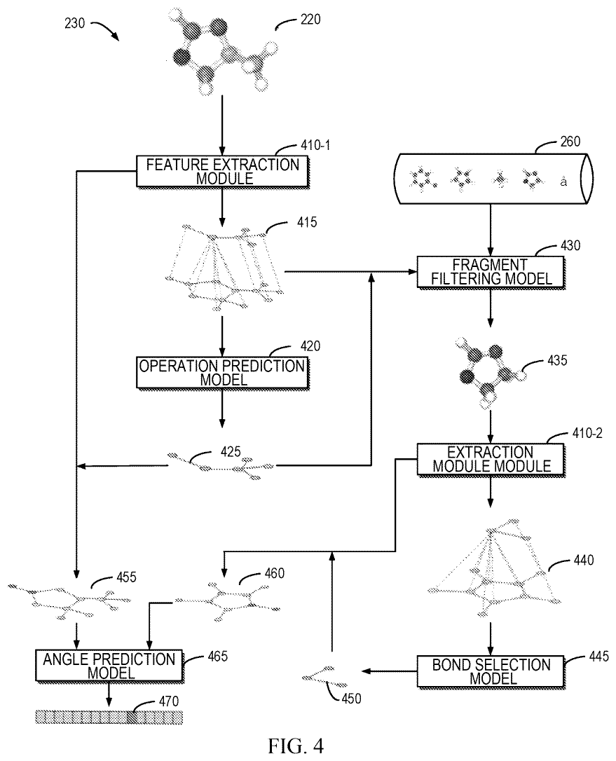

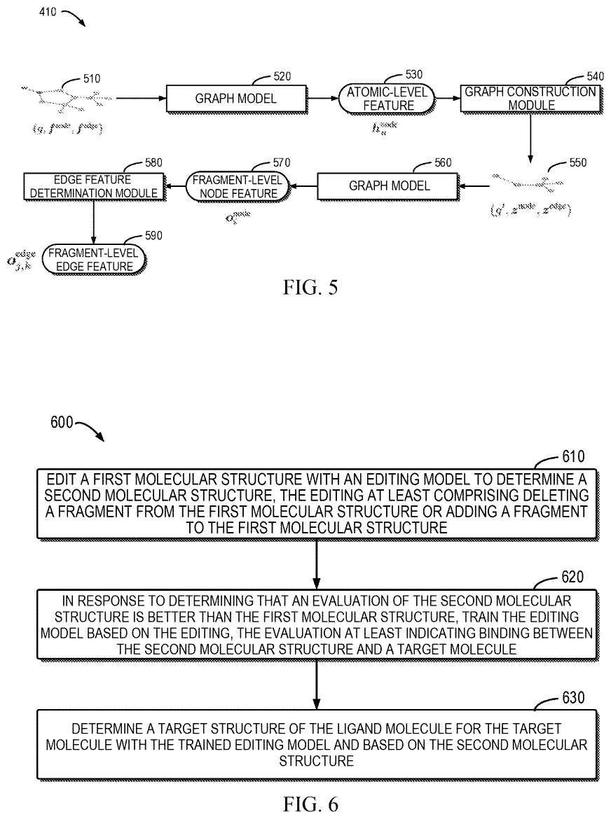

As shown in FIG. 2, the editing Module 230 may utilize the deployed editing model to edit the first molecular structure 220 to obtain the second molecular structure 250. The editing model may be implemented, for example, based on a Machine Learning model. Specific details regarding the editing Module 230 and the editing model will be described in detail below with reference to FIGS. 4 and 5.

As shown in FIG. 2, the design module 240 may also include a training module 240. In some embodiments, the training module 240 may determine an evaluation (also referred to as a first evaluation for convenience of description) for the second molecular structure 250. For example, the training module 240 may determine the first evaluation based on binding capacity between the second molecular structure 250 and the target molecule 170. Additionally, the training module 240 may also determine the first evaluation based on factors such as drug-like QED and/or synthesizability.

Furthermore, the training module 240 may compare the first evaluation of the second molecular structure 250 with the second evaluation of the first molecular structure 220. It should be understood that the training module 240 may determine the second evaluation regarding the first molecular structure based on a similar process. If it is determined that the first evaluation is better than the second evaluation, the training module 240 may utilize the editing operation performed by the editing module 230 to train the editing model deployed in the editing module 230.

In some embodiments, the editing module 230 may utilize a trained editing model and iteratively perform the editing based on the second molecular structure 250, until the target structure of the ligand molecule for the target molecule 170 is determined.

In some embodiments, the editing module 230 may, for example, terminate the iteration after editing the initial molecular structure 210 a predetermined number of times, and the final output of the molecular structure is determined as a target structure of the ligand molecule.

In some embodiments, the editing module 230 may also determine whether to converge based on the degree of change in the evaluation of the molecular structure edited after each iteration. For example, if the change in the evaluation after a predetermined number of iterations is less than a predetermined threshold, the editing module 230 may determine that it has converged and determine the final output molecular structure as the target structure of the ligand molecule.

The detailed process of self-supervised training will be described in detail below.

### Fragment Construct

As discussed above, the fragment library 260 may include the plurality of fragments 270. In some embodiments, the plurality of fragments 270 may be determined, for example, based on experimental knowledge, e.g., some known fragments of drug molecules.

In some embodiments, the fragments 270 in the fragment library 260 may also be constructed from known drug molecules. FIG. 3 shows a schematic block diagram of a fragment constructing module 300 in accordance with some embodiments of the present disclosure. It should be understood that the fragment constructing module 300 may be implemented by the computing device 100 where the design module 125 is deployed or by a different computing device.

As shown in FIG. 3, the fragment constructing module 300 may include a splitting module 320, which is configured to split the structure of one or a plurality of known drug molecules 310. In some embodiments, the splitting module 320 may identify single bonds in a two-dimensional (2D) structure of the drug molecule 310. Further, the splitting module 320 may split the drug molecule 310 into a plurality of 2D fragments 330 by breaking the single bonds in the 2D structure of the drug molecule 310. As shown in FIG. 3, for each split single bond, the splitting module 320 may correspondingly add a hydrogen atom to maintain charge balance.

In some embodiments, the fragment constructing module 300 may also include a generation module 340 configured to generate corresponding 3D fragments 270 based on a plurality of split 2D fragments 330. For example, the generation module 340 may utilize tools such as RDKit to generate corresponding 3D segments 270 based on 2D segments 330. In some embodiments, a 2D fragment 330 may correspond to a plurality of configurations, and the generation module 340 may add the corresponding plurality of configurations to the fragment library 260. Thus, it can be ensured that the added fragments are always rigid.

In some embodiments, the generation module 340 may also mark the bonds broken by the module 320 as editable bonds for use in the editing process in the editing module 230.

By breaking the single bonds in the drug molecule that lead to structural selectivity, the fragment constructing module 300 can ensure that the generated 3D fragments 270 are rigid. Therefore, in the add editing operation, the embodiments of the present disclosure only need to determine a dihedral angle of the two structural combinations, effectively simplifying the problem of molecular structure generation.

In addition, by using a fragment library including a plurality of 3D fragments to construct drug molecules, the embodiments of the present disclosure can directly construct the 3D structure of ligand molecules. Compared with traditional 2D structure generation methods, the embodiments of the present disclosure can further consider the influence between different 3D structures. In addition, by directly generating 3D molecular structures, the embodiments of the present disclosure can make them more intuitive and easier to understand.

### Molecular Structure Editing

As discussed with reference to FIG. 2, the editing module 230 is configured to edit the first 2D molecular structure 220 with the deployed editing model. FIG. 4 illustrates a schematic block diagram of an editing module 230 according to some embodiments of the present disclosure.

As shown in FIG. 4, the editing module 230 includes a feature extraction module 410-1, which is configured to generate fragment-level features 415 based on the first molecular structure 220. In some embodiments, the feature extraction module 410-1 may include a double-layer MPNN (Message Passing Neural Network), which can be used to generate atomic-level features and fragment-level features of the first molecular structure 220. Exemplarily, the processing of the feature extraction module 410-1 can be represented as:

$${h}^{node} , {o}^{node} , {o}^{edge} = HierMPNN ( {x}_{skel} ) \qquad ( 1 )$$

wherein, HierMPNN ( ) represents the operation of the feature extraction module 410-1, h^{node} represents atomic-level feature (also known as atomic-level node feature), o^{node} represents fragment-level node feature, o^{node} represents fragment-level edge feature, and x^{skel} represents the first molecular structure 220.

In some embodiments, the atomic-level node feature h^{node} may correspond to each atom in the first molecular structure 220, the fragment-level node feature o^{node} may correspond to each fragment in the first molecular structure 220, and the fragment-level edge feature o^{edge} may correspond to bonds among fragments in the first molecular structure 220. The implementation details of the feature extraction module 410-1 will be described in detail below with reference to FIG. 5.

As shown in FIG. 4, the editing module 230 includes an operation prediction model 420, which is configured to determine the target bond to be edited from a set of editable bonds based on a set of feature representations (also referred to as a first set of feature representations) of a set of editable bonds in the first molecular structure 220, and determines the editing operation to be applied to the target bond.

Illustratively, the processing process of the operation prediction model 420 may be represented as:

$${P}_{add} ( {x}_{skel} ) = Softmax ( \{ {MLP}_{1} ( {o}_{b}^{edge} ) \} b ∈ {V}_{a} ) \qquad ( 2 )$$

$${P}_{delete} ( {x}_{skel} ) = Softmax ( \{ {MLP}_{1} ( {o}_{b}^{edge} ) \} b ∈ {V}_{d} ) \qquad ( 3 )$$

wherein, MLP1 ( ) represents the operation of the operation prediction model 420, Vₐ represents the set of editable bonds in the first molecular structure 220 that can perform the add editing operation, and V_{d} represents the set of editable bonds in the first molecular structure 220 that can perform the delete editing operation. p_{add}(xₛₖₑₗ) represents the probability of performing the add editing operation on the first molecular structure 220 and p_{delete}(xₛₖₑₗ) represents the probability of performing the delete editing operation on the first molecular structure 220.

In some embodiments, the operation prediction model 420 may sample the determined target bond 425 to be edited and the editing operation applied to the target bond 425 based on the determined probability p_{add}(xₛₖₑₗ) and p_{delete}(xₛₖₑₗ). If the editing operation is determined to be a delete editing operation, the editing module 230 may correspondingly delete fragments associated with the target bond 425 from the first molecular structure 220, thereby obtaining the second molecular structure 250.

Alternatively, if it is determined that the editing operation applied to the target bond 425 is an add editing operation, the editing module 230 may further utilize a fragment filtering model 430 to determine fragments to be added to the first molecular structure 220.

As shown in FIG. 4, the fragment filtering model 430 may obtain a fragment-level edge feature (also referred to as a second feature representation) corresponding to the determined target bond 425 and determine the target fragment 435 in the fragment library 260 based on the second feature representation.

Illustratively, the processing process of the fragment filtering model 430 may be represented as:

$${p}_{fragment} ( {x}_{skel} , r ) = Softmax ( {MLP}_{2} ( {o}_{r}^{edge} ) ) \qquad ( 4 )$$

wherein, r represents the target bond 425, oᵣ^{edge} represents the fragment-level edge feature of target bond 425. MLP2 ( ) represents the operation of fragment filtering model 430, p_{fragment}(xₛₖₑₗ, r) represents the probability that each fragment in fragment library 260 is selected for addition to the target bond 425, where the probability of each dimension corresponds to a fragment in fragment library 260. As shown in FIG. 4, for example, the fragment filtering model 430 samples the target fragment 435 from the fragment library 260 based on the determined probability p_{fragment}(xₛₖₑₗ, r).

Further, the editing module 230 also includes a feature extraction module 410-2, which is configured to obtain a feature representation 440 of the target fragment 430. The feature extraction module 410-2 has the same structure as the feature extraction module 410-1, which is configured to generate atomic-level features and fragment-level features of the target fragment 430.

Illustratively, the processing process of the second feature extraction module 410-2 may be expressed as:

$${h}^{frag - node} , {o}^{frag - node} , {o}^{frag - edge} = HierMPNN ( {x}_{frag} ) \qquad ( 5 )$$

wherein, HierMPNN ( ) represents the operation of the feature extraction module 410-2 h^{frag-node} represents atomic-level feature, o^{frag-node} represents fragment-level node feature, o^{frag-edge} represents fragment-level edge feature, and x_{frag} represents the target fragment 435.

In some embodiments, the atomic-level node feature h^{frag-node} may correspond to each atom in the target fragment 435, the fragment-level node feature o^{frag-node} may correspond to each sub-fragment in the target fragment 435, and the fragment-level edge feature o^{frag-edge} may correspond to bonds among the sub-fragments in the target fragment 435. Here, the sub-fragments in the target fragment 435 represent one or more sub-fragments unsealed based on editable bonds in the target fragment 435.

In some embodiments, the editing module 230 further includes a bond selection model 445, which is configured to obtain a set of feature representations (for convenience of description, referred to as a set of third feature representations) in the target fragment that can be used to attach to a set of candidate bonds of the first molecular structure 220. Furthermore, the bond selection model 445 may also determine a fourth feature representation based on the first molecular structure 220 and the third feature representation.

Specifically, the bond selection model 445 may calculate the node mean feature based on the fragment-level node representation o^{node} of the first molecular structure 220:

$${\overline{o}}^{node} = MeanPool ( {o}^{node} ) \qquad ( 6 )$$

wherein, MeanPool ( ) represents mean pooling operation.

Further, the bond selection model 445 may cascade the third feature representation and the node mean feature to determine the fourth feature representation Concat(ō^{node}, o_{b}^{frag-edge}) where b represents a number of candidate bonds.

Illustratively, the bond selection model 445 may further determine an added bond from a set of candidate bonds based on a fourth feature representation, the processing process of which may be expressed as:

$${p}_{attach} ( {x}_{skel} , r , {x}_{frag} ) = Softmax ( {\{ {MLP}_{3} ( Concat ( {\overline{o}}^{node} , {o}_{b}^{frag - edge} ) ) \}}_{b = 1}^{{m}_{{x}_{frag}}} ) \qquad ( 7 )$$

wherein, MLP3 ( ) represents the calculation process of the bond selection model 445, mₓ_{_{frag}} represents the number of candidate bonds in the target fragment 435, and p_{attach}(xₛₖₑₗ, r, x_{frag}) represents the probability of each candidate bond being selected as an added bond.

In some embodiments, based on the determined probability p_{attach}(xₛₖₑₗ, r, x_{frag}), the bond selection model 445 may determine the added bond 450 for performing the add editing operation from a set of candidate bonds from the target fragment 435.

Further, as shown in FIG. 4, the editing module 230 further includes an angle prediction model 465 configured to determine a first pair of atoms in the first molecular structure 220 associated with the target bond 425 to be edited, and a second pair of atoms in the target fragment associated with the added bond 450.

In some embodiments, the angle prediction model 465 may obtain a first atomic-level feature representation 455 (i.e., h^{node}) of the first molecular structure 220 and a second atomic-level feature representation 460 (i.e., h^{frag-node}) of the target fragment 435. Furthermore, the angle prediction model 465 may determine the feature representation (also known as the fifth feature representation) corresponding to a pair of atoms uₛₖₑₗ and wₛₖₑₗ associated with the target bond 425 based on the first atomic level feature representation 455 and may determine the feature representation corresponding to a pair of atoms u_{frag} and w_{frag} associated with the added bond 450 based on the second atomic-level feature representation 460.

Further, the angle prediction model 465 may determine the first molecular structure 220 and the dihedral angle of the target fragment 435 based on a cascade of feature representations of four atoms, the processing process of which may be expressed as:

$${h}^{angle} = Concat ( {h}_{{u}_{skel}}^{node} , {h}_{{w}_{skel}}^{node} , {h}_{{u}_{frag}}^{frag - node} , {h}_{{w}_{frag}}^{frag - node} ) \qquad ( 8 )$$

$${P}_{angle} ( {x}_{skel} , r , {x}_{frag} , a ) = Softmax ( {MLP}_{4} ( {h}^{angle} ) ) \qquad ( 9 )$$

wherein, a represents the determined added bond 450, p_{angle}(xₛₖₑₗ, r, x_{frag}, a) represents the probability that the corresponding angle or angle range (e.g., 10 angle ranges, each range with 36 degrees) is selected as the dihedral angle, and MLP4 ( ) represents the calculation process of the angle prediction model 465.

In some embodiments, based on the determined probability p_{angle}(xₛₖₑₗ, r, x_{frag}, a), the bond selection model 445 may sample from a predetermined angle or angle range to determine a dihedral angle 470 for performing the add editing operation.

After determining the target bond 425 to be edited in the first molecular structure 220, the target fragment 435 for adding to the first molecular structure 220, the added bond 450 in the target fragment 435 and the dihedral angle 470 of the first molecular structure 220 and the target fragment 435, the editing module 230 may generate the second molecular structure 250 accordingly.

### Feature Extraction

Specific implementations of the feature extraction modules 410-1 and 410-2 (individually or collectively referred to as the feature extraction module 410) discussed in FIG. 4 will be described in detail below with reference to FIG. 5, which shows a schematic block diagram of the feature extraction module 410 in accordance with some embodiments of the present disclosure.

As shown in FIG. 5, the feature extraction module 410 includes a graph model 520 (also referred to as a first graph model 520), which is configured to obtain a graph 510 (also referred to as a first graph 510). The first graph 510 may be generated accordingly based on the molecular structure to be processed, which may be expressed, for example, as follows: x=(g, f^{node}, f^{edge}), where g represents the topological relationship of the first graph 510; f^{node} is a node in the first graph 510, which represents the features of each atom in the molecular structure; f^{edge} is an edge in the first graph 510, which represents the features of the bonds in the molecular structure.

Furthermore, the first graph model 520 may determine the corresponding atomic-level feature 530 based on the input first graph 510. Illustratively, the first graph model 520 may be an MPNN, and its processing process may be represented as:

$${h}_{u}^{node} = {{MPNN}_{1} ( g , {f}^{node} , {f}^{edge} )}_{u} ∈ {ℝ}^{d} \qquad ( 10 )$$

wherein, hᵤ^{node} represents the atomic-level feature of the node, which corresponds to an atom in the molecular structure.

It should be appreciated that the atomic-level feature h^{node} of the first molecular structure 220 and the atomic feature h^{frag-node} of the target fragment 435 discussed above with reference to FIG. 4 may be generated accordingly based on Equation (10).

As shown in FIG. 5, the feature extraction module 410 further includes a graph constructing module 540, which is configured to construct a second graph 550 based on atomic-level features 530. Specifically, the graph constructing module 540 may determine the node features (also referred to as a first set of node features) of each node in the second graph 550 based on the atomic-level features 530 of all atoms in each fragment of the molecular structure:

$${𝓏}_{j , k}^{node} = {MeanPool}_{u ∈ {V}_{i}} ( {h}_{u}^{node} ) ∈ {ℝ}^{d} \qquad ( 11 )$$

wherein, represents the collection of atoms in a fragment (or sub-fragment) of molecular structure.

Furthermore, the graph constructing module 540 may construct a set of edge features (predicted to be a first set of edge features) corresponding to a set of bonds among a set of fragments based on a set of node features corresponding to a set of fragments.

$${𝓏}_{j , k}^{edge} = {A}_{1} \cdot Concat ( {h}_{j}^{node} , {h}_{k}^{node} ) + {b}_{1} ∈ {ℝ}^{d} \qquad ( 12 )$$

wherein A₁ and b₁ may be pre-configured static parameters, configurable hyperparameters, or model parameters to be trained.

Further, the graph constructing module 540 may be based on a first set of node features and a first set of edge features to construct a second graph 550, which may be represented, for example, (g′, z^{node}, z^{edge}).

As shown in FIG. 5, the feature extraction module 410 further includes a graph model 560 (also referred to as a second graph model 560), which is configured to determine the corresponding fragment-level node feature 570 based on the input second graph 550. For example, the second graph model 560 may be MPNN, and its processing process may be represented as:

$${o}_{i}^{node} = {{MPNN}_{2} ( {g}^{′} , {𝓏}^{node} , {𝓏}^{edge} )}_{i} ∈ {ℝ}^{d} \qquad ( 13 )$$

wherein, oᵢ^{node} represents the fragment-level node feature, which corresponds to a fragment (or sub-fragment) in the molecular structure.

It should be appreciated that the fragment node level feature o^{node} of the first molecular structure 220 and the fragment-level node feature o^{frag-node} of the target fragment 435 discussed above with reference to FIG. 4 may be generated accordingly based on Equation (13).

As shown in FIG. 5, the feature extraction module 410 further includes an edge feature determination module 580 configured to determine a fragment-level edge feature 590 corresponding to bonds among a set of fragments (or sub-fragments) based on a fragment-level node feature 570:

$${o}_{j , k}^{edge} = {A}_{2} \cdot Concat ( {o}_{j}^{node} , {o}_{k}^{node} ) + {b}_{2} ∈ {ℝ}^{d} \qquad ( 14 )$$

wherein A₂ and b₂ may be pre-configured static parameters, configurable hyperparameters, or model parameters to be trained.

It should be appreciated that the fragment-level edge features o^{edge} of the first molecular structure 220 and the fragment-level edge features o^{frag-edge} of the target fragment 435 discussed above with reference to FIG. 4 may be generated accordingly based on Equation (13).

### Self-Supervised Training

In some embodiments, as discussed above with reference to FIG. 2, the training module 240 may self-supervised train the editing model based on the editing operation applied to the first molecular structure 220.

As discussed above, the target bond 425 to be edited in the first molecular structure 220, the target fragment 435 for adding to the first molecular structure 220, the added bond 450 in the target fragment 435 and the dihedral angle 470 of the first molecular structure 220 and the target fragment 435 are sampled based on the probability.

In some embodiments, the design module 125 may, for example, perform a plurality of samples in parallel to obtain a plurality of candidate molecular structures based on the first molecular structure 220. In some embodiments, the training module 240 may determine an evaluation for each candidate molecular structure. As discussed above, this evaluation may be based, for example, on the binding capacity between the candidate molecular structure and the target molecule 170, the drug-like QED (Quantitative Estimate of Drug-likeness) of the candidate molecular structure, and/or the synthesizability of the candidate molecular structure.

Illustratively, the training module 240 may determine the evaluation of each candidate molecular structure based on:

$$f ( x ) = binding energy ( x ) + {w}_{1} log QED ( x ) + {w}_{2} log SAscore ( x ) \qquad ( 15 )$$

wherein, x represents the candidate molecular structure, binding energy(x) represents the binding energy between the candidate molecular structure and the target molecule 170, QED(x) represents the drug-like property score of the candidate molecular structure, and SAscore(x) represents the synthesizability score of the candidate molecular structure. In addition, w₁ and w₂ are weight coefficients which may be pre-configured static parameters, configurable hyperparameters, or model parameters to be trained.

In some embodiments, the design module 125 may compare the evaluation of the candidate molecular structure with the evaluation of the first molecular structure 220 and determines whether the candidate molecular structure may be used as a basic structure for further editing.

In some embodiments, if the candidate molecular structure evaluation better than the evaluation of the first molecular structure 220, the design module 125 may determine that the candidate molecular structure may be used as the basic structure for the next editing.

In some embodiments, if the evaluation of the candidate molecular structure is worse than or equal to the evaluation of the first molecular structure, the design module 125 may determine the probability that the candidate molecular structure is selected as the basic structure for the next editing:

$$min \{ 1 , exp ( \frac{f ( {x}^{′} ) - f ( x )}{T} ) \} \qquad ( 16 )$$

wherein f(x′) represents the evaluation of the candidate molecular structure, f(x) represents the evaluation of the first molecular structure 220, T represents a temperature coefficient, which may be determined, for example, based on the number of editing operations applied from the initial molecular structure 210 to the candidate molecular structure.

In this way, some editing operations that lead to reduced evaluation may also be randomly retained, thereby improving the diversity of drug molecule generation.

In some embodiments, for the candidate molecular structure with the evaluation better than the first molecular structure 220, the training module 240 may further train the editing model based on the editing operation corresponding to the generated candidate molecular structure.

In some embodiments, the training editing model may be based on a weighted maximum likelihood estimate (WMLE), whose optimization objectives for training may be determined, for example:

$${max}_{θ} \frac{1}{\vert D \vert} {\sum }_{( x , {x}^{′} ) ∈ D} λ ( {x}^{′} - x ) log {q}_{θ} ( {x}^{′} \vert x ) \qquad ( 17 )$$

wherein, D represents all the constructed pairs (x,x′) constructed by a candidate molecular structure obtained better evaluation and the first molecular structure 220, λ(x′,x) is a monotonic function that is positively correlated with the evaluation difference between the candidate molecular structure and the first molecular structure 220.

By using WMLE, the embodiments of the present disclosure can greatly reduce the deviation of gradient descent. In addition, through self-supervised training, the embodiments of the present disclosure can get rid of the dependence on experimental data, thereby improving the universality of drug design methods.

### Example Process

FIG. 6 shows a flowchart of a method of designing ligand molecules 600 implemented in accordance with some of the present disclosure. The method 600 may be implemented by computing device 100, for example, at the design module 125 in the memory 620 of the computing device 600.

As shown in FIG. 6, at block 610, the computing device 100 edits a first molecular structure with an editing model to determine a second molecular structure, the editing at least comprising deleting a fragment from the first molecular structure or adding a fragment to the first molecular structure.

At block 620, in response to determining that an evaluation of the second molecular structure is better than the first molecular structure, the computing device 100 trains the editing model based on the editing, the evaluation at least indicating binding capacity between the second molecular structure and a target molecule.

At block 630, the computing device 100 determines a target structure of the ligand molecule for the target molecule with the trained editing model and based on the second molecular structure.

The following are some example implementations of the present disclosure.

In some embodiments, the editing model comprises an operation prediction model, and wherein editing a first molecular structure with an editing model comprises: determining, based on the first molecular structure, a set of first feature representations of a set of editable bonds in the first molecular structure; determining a target bond to be edited from the set of editable bonds and determining an editing operation to be applied to the target bond with the operation prediction model and based on the set of first feature representations; and editing the first molecular structure based on the determined editing operation.

In some embodiments, editing a first molecular structure comprising adding a fragment to the first molecular structure comprises: selecting a target fragment from a fragment library, wherein the fragment library comprises a plurality of three-dimensional fragments; and adding the target fragment to the first molecular structure.

In some embodiments, the plurality of three-dimensional fragments are constructed based on breaking single bonds in a set of drug molecules.

In some embodiments, the editing model comprises a fragment filtering model, and selecting a target fragment from a fragment library comprises: determining, based on the first molecular structure, a second feature representation of a target bond to be edited in the first molecular structure; and determining the target fragment from the fragment library with the fragment filtering model and based on the second feature representation.

In some embodiments, adding the target fragment to the first molecular structure comprises: determining an added bond added to the first molecular structure in the target fragment; and adding the target fragment to the first molecular structure based on the added bond.

In some embodiments, the editing model comprises a bond selection model, and wherein determining an added bond added to the first molecular structure in the target fragment comprises: determining a set of third feature representations of a set of candidate bonds that are capable of being used to add to the first molecular structure in the target fragment; determining a fourth feature representation based on the first molecular structure and the third feature representation; and determining the added bond from the set of candidate bonds with the bond selection model and based on the fourth feature representation.

In some embodiments, adding the target fragment to the first molecular structure based on the added bond comprises: determining a first pair of atoms associated with a target bond to be edited in the first molecular structure; determining a second pair of atoms associated with the added bond in the target fragment; determining a dihedral angle for adding the target fragment based on the first pair of atoms and the second pair of atoms; and adding the target fragment to the first molecular structure based on the determined dihedral angle.

In some embodiments, the editing model comprises an angle prediction model, and determining a dihedral angle for adding the target fragment comprises: determining a fifth feature representation associated with the first pair of atoms and the second pair of atoms based on the first molecular structure and the target fragment; and determining the dihedral angle for adding the target fragment with the angle prediction model and based on the fifth feature representation.

In some embodiments, editing a first molecular structure comprising deleting a fragment from the first molecular structure comprises: determining a bond to be deleted in the first molecular structure; and deleting a fragment associated with the bond to be deleted from the first molecular structure.

In some embodiments, determining a target structure of the ligand molecule for the target molecule comprises: processing the second molecular structure with the trained editing model to determine a third molecular structure; and determining the target structure of the ligand molecule based on the third molecular structure.

In some embodiments, the first molecular structure is generated by applying the first number of editing operations to an initial molecular structure, and determining a target structure of the ligand molecule for the target molecule comprises: incrementing the first number to determine the second number; and if the second number reaches a predetermined threshold, determining the second molecular structure as the target structure.

In some embodiments, the method further comprising: in response to determining that the evaluation of the second molecular structure is worse than or equal to the first molecular structure, determining, based on the evaluation, a probability that the second molecular structure is used to determine the target structure of the ligand molecule.

In some embodiments, the first molecular structure is generated by applying the first number of editing operations to an initial molecular structure, and the probability is further based on the first number.

In some embodiments, the evaluation is a first evaluation and training the editing model based on the editing comprises: training the editing model based on an optimization objective to be determined based on a difference between the first evaluation and a second evaluation of the first molecular structure.

In some embodiments, the editing model comprises a first graph model, and wherein editing a first molecular structure with an editing model comprises: generating a first graph based on the first molecular structure, wherein a first set of nodes in the first graph corresponds to a set of atoms in the first molecular structure, and a first set of edges in the first graph corresponds to a set of bonds in the first molecular structure; processing the first graph with the first graph model to determine a set of atomic-level features corresponding to the set of atoms; and editing the first molecular structure based on the set of atomic-level features.

In some embodiments, the editing model further comprises a second graph model, and editing the first molecular structure based on the set of atomic-level features comprises: determining, based on the set of atomic-level features, a first set of node features corresponding to a set of fragments in the first molecular structure and a first set of edge features corresponding to a set of bonds among the set of fragments; constructing a second graph based on the first set of node features and the first set of edge features; processing the second graph with the second graph model to determine a set of fragment-level node features corresponding to the set of fragments; and editing the first molecular structure based on at least one of the set of fragment-level node features or the set of atomic-level features.

In some embodiments, editing the first molecular structure based on at least one of the set of fragment-level node features or the set of atomic-level features comprises: determining, based on the set of fragment-level node features, a set of fragment-level edge features corresponding to a set of bonds among the set of fragments; and editing the first molecular structure based on at least one of the set of fragment-level node features, the set of atomic-level features, or the set of fragment-level edge features.

In some embodiments, the evaluation is further based on: drug-like QED of the second molecular structure; or synthesizability of the second molecular structure.

Herein, the functions described above may be at least partially performed by one or more hardware logic components. For example, without limitation, exemplary types of hardware logic components that may be used include: field programmable gate arrays (FPGAs), application specific integrated circuits (ASICs), application specific standard products (ASSPs), system on a chip (SOC), load programmable logic devices (CPLDs), and the like.

The program code for implementing the methods disclosed herein may be written in any combination of one or a plurality of programming languages. The program code may be provided to a processor or controller of a general-purpose computer, special purpose computer, or other programmable data processing apparatus such that the program code, when executed by the processor or controller, causes the functions/operations specified in the flowcharts and/or block diagrams to be implemented. The program code may be executed entirely on the machine, partially on the machine, partially as a standalone software package, and partially on a remote machine or entirely on a remote machine or server.

In the context of this disclosure, a machine-readable medium may be a tangible medium that may contain or store a program for use by or in connection with an instruction execution system, apparatus, or device. The machine-readable medium may be a machine-readable signal medium or a machine-readable storage medium. The machine-readable medium may include, but is not limited to, electronic, magnetic, optical, electromagnetic, infrared, or semiconductor systems, devices, or devices, or any suitable combination thereof. More specific examples of machine-readable storage media may include electrical connections based on one or a plurality of wires, portable computer disks, hard disks, random access memory (RAM), read-only memory (ROM), erasable programmable read-only memory (EPROM or flash memory), optical fibers, portable compact disc read-only memory (CD-ROM), optical storage devices, magnetic storage devices, or any suitable combination thereof.

In addition, although operations are depicted in a specific order, it should be understood that such operations are required to be performed in the specific order shown or in sequential order, or that all illustrated operations should be performed to achieve the desired result. Under certain circumstances, multitasking and parallel processing may be advantageous. Similarly, although several specific implementation details are included in the above discussion, these should not be construed as limiting the scope of the present disclosure. Certain features described in the context of individual implementations can also be implemented in combination in a single implementation. Conversely, various features described in the context of a single implementation can also be implemented separately or in any suitable sub-combination in a plurality of implementations.

Although the subject matter has been described in language specific to structural features and/or methodological logical acts, it should be understood that the subject matter defined in the appended claims is not necessarily limited to the particular features or acts described above. Rather, the particular features and acts described above are merely exemplary forms of implementation of the claims.

1: A method of designing ligand molecules, comprising:
editing a first molecular structure with an editing model to determine a second molecular structure, the editing at least comprising deleting a fragment from the first molecular structure or adding a fragment to the first molecular structure;
in response to determining that an evaluation of the second molecular structure is better than the first molecular structure, training the editing model based on the editing, the evaluation at least indicating binding capacity between the second molecular structure and a target molecule; and
determining a target structure of the ligand molecule for the target molecule with the trained editing model and based on the second molecular structure.

2: The method of claim 1, wherein the editing model comprises an operation prediction model, and wherein editing a first molecular structure with an editing model comprises:
determining, based on the first molecular structure, a set of first feature representations of a set of editable bonds in the first molecular structure;
determining a target bond to be edited from the set of editable bonds and determining an editing operation to be applied to the target bond with the operation prediction model and based on the set of first feature representations; and
editing the first molecular structure based on the determined editing operation.

3: The method of claim 1, wherein editing a first molecular structure comprising adding a fragment to the first molecular structure comprises:
selecting a target fragment from a fragment library, wherein the fragment library comprises a plurality of three-dimensional fragments; and
adding the target fragment to the first molecular structure.

4: The method of claim 3, wherein the plurality of three-dimensional fragments are constructed based on breaking single bonds in a set of drug molecules.

5: The method of claim 3, wherein the editing model comprises a fragment filtering model, and selecting a target fragment from a fragment library comprises:
determining, based on the first molecular structure, a second feature representation of a target bond to be edited in the first molecular structure; and
determining the target fragment from the fragment library with the fragment filtering model and based on the second feature representation.

6: The method of claim 3, wherein adding the target fragment to the first molecular structure comprises:
determining an added bond added to the first molecular structure in the target fragment; and
adding the target fragment to the first molecular structure based on the added bond.

7: The method of claim 6, wherein the editing model comprises a bond selection model, and wherein determining an added bond added to the first molecular structure in the target fragment comprises:
determining a set of third feature representations of a set of candidate bonds that are capable of being used to add to the first molecular structure in the target fragment;
determining a fourth feature representation based on the first molecular structure and the third feature representation; and
determining the added bond from the set of candidate bonds with the bond selection model and based on the fourth feature representation.

8: The method of claim 6, wherein adding the target fragment to the first molecular structure based on the added bond comprises:
determining a first pair of atoms associated with a target bond to be edited in the first molecular structure;
determining a second pair of atoms associated with the added bond in the target fragment;
determining a dihedral angle for adding the target fragment based on the first pair of atoms and the second pair of atoms; and
adding the target fragment to the first molecular structure based on the determined dihedral angle.

9: The method of claim 8, wherein the editing model comprises an angle prediction model, and determining a dihedral angle for adding the target fragment comprises:
determining a fifth feature representation associated with the first pair of atoms and the second pair of atoms based on the first molecular structure and the target fragment; and
determining the dihedral angle for adding the target fragment with the angle prediction model and based on the fifth feature representation.

10: The method of claim 1, wherein editing a first molecular structure comprising deleting a fragment from the first molecular structure comprises:
determining a bond to be deleted in the first molecular structure; and
deleting a fragment associated with the bond to be deleted from the first molecular structure.

11: The method of claim 1, wherein determining a target structure of the ligand molecule for the target molecule comprises:
processing the second molecular structure with the trained editing model to determine a third molecular structure; and
determining the target structure of the ligand molecule based on the third molecular structure.

12: The method of claim 1, wherein the first molecular structure is generated by applying the first number of editing operations to an initial molecular structure, and determining a target structure of the ligand molecule for the target molecule comprises:
incrementing the first number to determine the second number; and
if the second number reaches a predetermined threshold, determining the second molecular structure as the target structure.

13: The method of claim 1, further comprising:
in response to determining that the evaluation of the second molecular structure is worse than or equal to the first molecular structure, determining, based on the evaluation, a probability that the second molecular structure is used to determine the target structure of the ligand molecule.

14: The method of claim 13, wherein the first molecular structure is generated by applying the first number of editing operations to an initial molecular structure, and the probability is further based on the first number.

15: The method of claim 1, wherein the evaluation is a first evaluation and training the editing model based on the editing comprises:
training the editing model based on an optimization objective to be determined based on a difference between the first evaluation and a second evaluation of the first molecular structure.

16: The method of claim 1, wherein the editing model comprises a first graph model, and wherein editing a first molecular structure with an editing model comprises:
generating a first graph based on the first molecular structure, wherein a first set of nodes in the first graph corresponds to a set of atoms in the first molecular structure, and a first set of edges in the first graph corresponds to a set of bonds in the first molecular structure;
processing the first graph with the first graph model to determine a set of atomic-level features corresponding to the set of atoms; and
editing the first molecular structure based on the set of atomic-level features.

17: The method of claim 16, wherein the editing model further comprises a second graph model, and editing the first molecular structure based on the set of atomic-level features comprises:
determining, based on the set of atomic-level features, a first set of node features corresponding to a set of fragments in the first molecular structure and a first set of edge features corresponding to a set of bonds among the set of fragments;
constructing a second graph based on the first set of node features and the first set of edge features;
processing the second graph with the second graph model to determine a set of fragment-level node features corresponding to the set of fragments; and
editing the first molecular structure based on at least one of the set of fragment-level node features or the set of atomic-level features.

18: The method of claim 17, wherein editing the first molecular structure based on at least one of the set of fragment-level node features or the set of atomic-level features comprises:
determining, based on the set of fragment-level node features, a set of fragment-level edge features corresponding to a set of bonds among the set of fragments; and
editing the first molecular structure based on at least one of the set of fragment-level node features, the set of atomic-level features, or the set of fragment-level edge features.

19. (canceled)

20. (canceled)

21: An electronic device, comprising:
a processor; and
a memory configured to store one or more computer instructions, the one or more computer instructions, when executed by the processor, implementing acts of designing ligand molecules, the acts comprising:
editing a first molecular structure with an editing model to determine a second molecular structure, the editing at least comprising deleting a fragment from the first molecular structure or adding a fragment to the first molecular structure;
in response to determining that an evaluation of the second molecular structure is better than the first molecular structure, training the editing model based on the editing, the evaluation at least indicating binding capacity between the second molecular structure and a target molecule; and
determining a target structure of the ligand molecule for the target molecule with the trained editing model and based on the second molecular structure.

22: A non-transitory computer-readable storage medium having one or more computer instructions stored thereon, the one or more computer instructions, when executed by a processor, implementing acts of designing ligand molecules, the acts comprising:
editing a first molecular structure with an editing model to determine a second molecular structure, the editing at least comprising deleting a fragment from the first molecular structure or adding a fragment to the first molecular structure;
in response to determining that an evaluation of the second molecular structure is better than the first molecular structure, training the editing model based on the editing, the evaluation at least indicating binding capacity between the second molecular structure and a target molecule; and
determining a target structure of the ligand molecule for the target molecule with the trained editing model and based on the second molecular structure.

23. (canceled)
